(19) United States
(12) Patent Application Publication
(10) Pub. No.: US 2018/0193193 A1
(43) Pub. Date: Jul. 12, 2018

(54) LACRIMAL DRAINAGE SYSTEM DIAGNOSTIC IMPLANT

(71) Applicant: THE REGENTS OF THE UNIVERSITY OF COLORADO, A BODY CORPORATE, Denver, CO (US)

(72) Inventor: Malik Y. KAHOOK, Denver, CO (US)

(73) Assignee: The Regents of the University of Colorado, A Body Corporate, Denver, CO (US)

(21) Appl. No.: 15/741,918

(22) PCT Filed: Jul. 6, 2016
(86) PCT No.: PCT/US2016/041136

§ 371 Date: Jan. 4, 2018

Related U.S. Application Data
(60) Provisional application No. 62/188,895, filed on Jul. 6, 2015.

(51) Int. Cl. A61F 9/007; A61F 9/00

(52) U.S. Cl. CPC A61F 9/00772; A61M 5/1723; A61F 9/0017

(57) ABSTRACT

A lacrimal drainage system diagnostic device for analyzing fluid from the eye may include a device body having an internal lumen and configured to be implanted in a lacrimal duct. A power source may be coupled to the device body. A control subsystem may be coupled to the power source. An analytical sensor may be within the internal lumen and in communication with the control subsystem.

### CROSS-REFERENCE TO RELATED APPLICATIONS

This application claims the benefit of U.S. Provisional Patent Application No. 62/188,895 filed Jul. 6, 2015 entitled “LACRIMAL DRAINAGE SYSTEM DIAGNOSTIC IMPLANT”, which is incorporated by reference herein in its entirety.

### FIELD OF THE INVENTION

The present application relates to medical diagnostics related to the lacrimal tear drainage system. In some embodiments, the present application relates to a lacrimal drainage system device and methods of using the device for tear analysis and treatment of related eye, sinuses and/or periocular tissue conditions.

### BACKGROUND OF THE INVENTION

Poor tear quality, including conditions such as dry eye disease (DED), can significantly reduce the quality of life of a subject. DED results from the disruption of the natural tear film on the surface of the eye, and can result in ocular discomfort, visual disturbance and a reduction in vision-related quality of life. Activities of daily living such as driving, computer use, housework and reading have been shown to be negatively impacted by DED and poor tear quality. Patients with severe cases of DED and poor tear quality are at risk for serious ocular health deficiencies such as corneal ulceration, and can experience a quality of life deficiency comparable to that of moderate-severe angina.

The etiology of DED is becoming increasingly well understood. DED is progressive in nature, and fundamentally results from insufficient tear coverage on the surface of the eye. This poor tear coverage prevents healthy gas exchange and nutrient transport for the ocular surface, promotes cellular desiccation and creates a poor refractive surface for vision. Poor tear coverage typically results from: 1) insufficient aqueous tear production from the lacrimal glands (e.g. secondary to post-menopausal hormonal deficiency, auto-immune disease, LASIK surgery, etc.), and/or 2) excessive evaporation of aqueous tear resulting from dysfunction of the meibomian glands. Low tear volume may cause a hyperosmolar environment that induces an inflamed state of the ocular surface. This inflammatory response may induce apoptosis of the surface cells which in turn may prevent proper distribution of the tear film on the ocular surface so that any given tear volume is rendered less effective. This initiates a vicious cycle where more inflammation can ensue causing more surface cell damage, etc. Additionally, the neural control loop, which controls reflex tear activation, may be disrupted because the sensory neurons in the surface of the eye are damaged. As a result, fewer tears are secreted and a second vicious cycle develops that may result in further progression of the disease (fewer tears cause nerve cell loss, which results in fewer tears, etc.).

In order to eye treat infection, inflammation of the eye, glaucoma and other ocular diseases or disorders, drugs are often required to be administered to the eye. A conventional method of drug delivery is by topical drop application to the eye's surface. Topical eye drops, though effective, can be inefficient. As one example, when an eye drop is instilled in an eye, it often overfills the conjunctival sac (i.e., the pocket between the eye and the lids) causing a substantial portion of the drop to be lost due to overflow of the lid margin and spillage onto the cheek. In addition, a large portion of the drop remaining on the ocular surface can be washed away into and through a lacrimal canaliculus, thereby diluting the concentration of the drug before it can treat the eye. Moreover, topically applied drugs often have a peak ocular effect for about two hours post-application, after which additional applications of the drugs should be, but are often not, administered to maintain the desired drug therapeutic benefit.

To compound ocular management difficulty, patients often do not use their eye drops as prescribed. This poor compliance can be due to, for example, an initial stinging or burning sensation caused by the eye drop and experience by a patient. Instilling eye drops in one's own eye can be difficult, in part because of the normal reflex to protect the eye. Therefore, one or more drops may miss the eye. Older patients may have additional problems instilling drops due to arthritis, unsteadiness, and decreased vision. Pediatric and psychiatric populations pose difficulties as well.

Conditions of DED have been treated by blocking the tear flow from the eye into and through the lacrimal canaliculus. This has involved closing the canalicular canal by stitching the punctal opening shut or by using electrical or laser cauterization to seal the punctal opening. Although such procedures can provide the desired result of blocking tear flow to treat a dry eye, they are unfortunately not reversible without reconstructive surgery.

While there is a wide spectrum of treatments for DED or poor tear quality, none provides substantial efficacy for treatment of the condition, nor do any of the current treatment or diagnostic analysis methods available provide real-time analysis of tears or induce appropriate treatment. Accordingly, it would be desirable to have a more effective, practical, and unobtrusive diagnostic analysis of tears to direct treatment for the tear condition.

### BRIEF SUMMARY OF THE INVENTION

In one embodiment, there is a device for analyzing fluid from the eye comprising a device body having an internal lumen and configured to be implanted in a lacrimal duct. A power source may be coupled to the device body. A control subsystem may be coupled to the power source. An analytical sensor may be within the internal lumen and in communication with the control subsystem. The internal lumen may include a proximal end with a proximal end sensor that includes an antenna for communicating with the control subsystem. In a further embodiment, the proximal end comprises a faceplate. The internal lumen may include a closed distal end. The internal lumen may be configured to impede the flow of fluid therethrough. The internal lumen may include at least one of a plug and a tapered internal lumen to impede the flow of fluid therethrough. The analytical sensor may comprise a tear analysis sensor. The tear analysis sensor may be configured to detect a parameter selected from the group consisting of amount of fluid, composition of molecules in tear fluid, osmolarity, osmolality, proteins, enzymes, medications, and antibodies. The analytical sensor may comprises at least one of a fluid volume sensor, an osmolarity sensor, an osmolality sensor, a chemical sensor, a conductivity sensor, a moisture sensor, a biomarker sensor, a molecular sensor, a biological sensor, and an electronic sensor. The control subsystem may comprise memory configured to store operational data from the device. The control subsystem may include an antenna having a radio frequency power amplifier. The device may be configured to traverse a user's nasolacrimal system through the user's lacrimal duct. The device may further comprise an in-line therapeutic agent reservoir in electronic communication with the analytical sensor and the control subsystem. In a further embodiment, the device includes an in-line therapeutic agent reservoir with a second device in electronic communication with the analytical sensor and the control subsystem of the device. The control subsystem may include logic communication circuits which receive specific stimulus programming instructions through an antenna in communication with the analytical sensor. The antenna may provide a wireless link between the control subsystem and a wireless device.

In one embodiment, a method for treating a condition of an eye of a subject comprises implanting a device for analyzing fluid from the eye into a user's lacrimal system such that an end of the device contacts a user's eye tear film. The device may include a device body having an internal lumen, a power source coupled to the device body, a control subsystem coupled to the power source, and an analytical sensor within the internal lumen and in communication with the control subsystem. The method may include evaluating fluid from the user's eye within the internal lumen with the analytical sensor to detect an eye condition. The eye condition may comprise at least one of dry eye, allergic response, inflammation, high glucose level, and low glucose level. The analytical sensor may detect a parameter selected from the group consisting of amount of fluid, composition of molecules in the tear fluid, osmolarity, osmolality, proteins, enzymes, and antibodies. The treating a symptom step may include providing medication to the user's eye from an in-line therapeutic agent reservoir.

### BRIEF DESCRIPTION OF THE SEVERAL VIEWS OF THE DRAWINGS

The foregoing summary, as well as the following detailed description of embodiments of the diagnostic implant, will be better understood when read in conjunction with the appended drawings of an exemplary embodiment. It should be understood, however, that the invention is not limited to the precise arrangements and instrumentalities shown.

In the drawings:

FIG. 1 is a side sectional side view of a typical person's eye including tear layers;

FIG. 2 is an illustration of a typical person's lacrimal system;

FIG. 3 is a front elevation view of a diagnostic device in accordance with a first exemplary embodiment of the present invention;

FIG. 4 is a front elevation view of a diagnostic device in accordance with a second exemplary embodiment of the present invention;

FIG. 5 is a front elevation view of the diagnostic device of FIG. 3 being inserted into a person's lacrimal system;

FIG. 6 is a front elevation view of the diagnostic device of FIG. 3 implanted in a person's lacrimal system; and

FIG. 7 is a flow chart depicting operation of the diagnostic device of FIG. 3.

### DETAILED DESCRIPTION OF THE INVENTION

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “patient” or “subject” refers to any living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As used herein, the terms “treat” and “treating” are not limited to the case where the subject (e.g. a patient) is cured and the disease is eradicated. Rather, treatment may also merely reduce symptoms, improves (to some degree) and/or delays disease progression among other effects. It is not intended that treatment be limited to instances wherein a disease or affliction is cured. It is sufficient that symptoms are reduced.

“Prevention” or “preventing” as used herein, includes, but is not limited to: (1) inhibiting the onset of a disease in a subject or patient which may be at risk and/or predisposed to the disease, wherein such inhibition may be either partial or complete, but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “ocular health” refers to restoring or maintaining the normal amount of tears in the eye to minimize dryness and related discomfort and to maintain eye health (including but not limited to treating or preventing at least one symptom associated with dry eye; stinging, burning or scratchy sensation in the eyes; stringy mucus in or around your eyes; increased eye irritation from smoke or wind; eye fatigue; sensitivity to light; eye redness; a sensation of having something in your eyes; difficulty wearing contact lenses; periods of excessive tearing; and blurred vision, often worsening at the end of the day or after focusing for a prolonged period).

As used herein, the terms “medication” or “therapeutic agent” refer to any compound and/or molecule that treats or prevents or alleviates the symptoms of disease or condition, including, but not limited to, a drug or pharmaceutical composition. Medication is considered to be delivered or present in therapeutically effective amounts or pharmaceutically effective amounts.

“Therapeutically effective amounts” or “pharmaceutically effective amounts”, as used herein, means that amount which, when administered to a subject or patient for treating a disease, is sufficient to effect such treatment for the disease or to ameliorate one or more symptoms of a disease or condition (e.g. ameliorate pain).

As used herein, the term “medication reservoir” or “therapeutic agent reservoir” refers to any structure containing medication or therapeutic agent. In preferred embodiments, the reservoir is made of stretchy plastics or silicones. In one embodiment, said reservoirs are selected from the reservoirs described by Yang, W.-W. and Pierstorff, E. (2012) “Reservoir-Based Polymer Drug Delivery Systems,” Journal of Laboratory Automation 17(1), 50-58; incorporated by reference herein.

As used herein, the term “medicament” refers to any active agent that is suitable for use in medical treatment, such as a medicinal compound or drug.

As used herein, the term “biomarker” refers to a measurable substance in an organism whose presence is indicative of some phenomenon such as disease, infection, or environmental exposure. Biomarkers are often measured and evaluated to examine normal biological processes, pathogenic processes, or pharmacologic responses to a therapeutic intervention.

As used herein, the term “active agent” refers to any molecular entity that exerts an effect on a living organism.

As used herein, the term “release of an agent” refers to any presence of the agent, or a subcomponent thereof, emanating from an implant device.

As used herein, the terms “analogue or analog” refer to any chemical compound that is structurally similar to a parent compound but differs slightly in composition (e.g., one atom or functional group is different, added, or removed). An analogue may or may not have different chemical or physical properties than the original compound and may or may not have improved biological and/or chemical activity. For example, the analogue may be more hydrophilic, or it may have altered reactivity as compared to the parent compound. The analogue may mimic the chemical and/or biological activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, the term “derivative” refers to any chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” An analogue may have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives that can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher, D. et al. (1996) “Improved Oral Drug Delivery: Solubility Limitations Overcome by the Use of Prodrugs,” Adv. Drug Delivery Rev. 19(2), 115-130, incorporated herein by reference. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds that simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

Any concentration ranges, percentage range, or ratio range recited herein are to be understood to include concentrations, percentages or ratios of any integer within that range and fractions thereof, such as one tenth and one hundredth of an integer, unless otherwise indicated. In addition, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to both one polymer or a mixture comprising two or more polymers.

As used herein, the term “biomaterial” refers to any substance (other than drugs) or combination of substances synthetic or natural in origin, which can be used for any period of time, as a whole or as a part of a system which treats, augments, or replaces any tissue, organ, or function of the body.

As used herein, the term “biocompatibility” refers to the ability of a material to perform with an appropriate host response in a specific application.

As used herein, the terms “medical device,” “implant,” “device,” “medical device,” “medical implant,” “implant/device,” and the like are used synonymously to refer to any object that is designed to be placed partially or wholly within a patient's body for one or more therapeutic or prophylactic purposes such as for tissue augmentation, tissue stimulation, contouring, restoring physiological function, repairing or restoring tissues damaged by disease or trauma, and/or delivering therapeutic agents to normal, damaged or diseased organs and tissues. While medical devices are normally composed of biologically compatible synthetic materials (e.g., medical-grade stainless steel, nitinol, titanium and other metals; exogenous polymers, such as polyurethane, silicone, PLA, PLGA, PGA, PCL), other materials may also be used in the construction of the medical implant. While not limiting the present invention to any particular device, specific medical devices and implants that are particularly relevant to this invention include stents, punctal plugs, Crawford tubes, catheters, lacrimal tubes, ocular or other shunts, and drug delivery systems. In some embodiments, the device incorporates a contrast material or opaque materials that allow for visualization with standard imaging devices (for example, barium to allow for x-ray visualization).

As used herein, the term “hydrogel” is used to refer to an absorbing or otherwise retaining material (e.g., adsorbing material), such as super-absorbent polymers, hydrocolloids, and water-absorbent hydrophilic polymers, for example. In some examples, the term “hydrogel” refers to super-absorbent polymer particles in a “dry or dehydrated” state, more specifically, particles containing from no water up to an amount of water less than the weight of the particles, such as less than about 5%, by weight, water. In some examples, the term “hydrogel” refers to a super-absorbent polymer in the “dry or dehydrated” state when the hydrogel is not expandable and also refers to its hydrated or expanded state, more specifically, hydrogels that have absorbed at least their weight in water, such as several times their weight in water. As the hydrogel material absorbs fluid, it size can increase and its shape can change to bias against at least a portion of a lacrimal canaliculus ampulla or lacrimal canaliculus wall, for example.

As used herein, the term “polymer” refers to any organic macromolecule containing one or more repeating units, as is well known in the art.

As used herein, a “copolymer” refers to any polymer in which there are at least two types of repeating units included. A copolymer can be a block copolymer, in which there are segments containing multiple repeating units of one type, bonded to segments containing multiple repeating units of a second type.

As used herein, the term “hydrophilic polymer” refers to any polymer that can be wetted by water, i.e., does not have a water-repellant surface. A hydrophilic polymer can absorb water to a small degree, for example about 0-100 wt % of water, but does not greatly swell in volume as does a hydrogel-forming polymer.

As used herein, the terms “implanted” refers to having completely or partially placed a device within a host. A device is partially implanted when some of the device reaches, or extends to the outside of, a host.

As used herein, the term “elastic limit” or “yield strength” refers to the stress at which a material begins to deform plastically. Prior to the yield point the material will deform elastically and will return to its original shape when the applied stress is removed. Once the yield point is passed, some fraction of the deformation will be permanent and non-reversible.

As used herein, the term “elastic” refers to a material that may have very large deformability when forces are applied on it with complete recoverability, meaning the object will return to its initial shape and size when these forces are removed. Such a feature has also been referred to as rubber elasticity. Such “elastic” materials may consist of polymer chains. Elastic materials may change conformation and extension under stress. Polymer chains may be highly flexible. Elastic materials may access conformational changes (not w/glassy, crystalline, stiff mat.) Polymer chains may be joined in a network structure. Elastic materials may avoid irreversible chain slippage (permanent strain). One out of 100 monomers may connect two different chains. Connections (covalent bond, crystallite, glassy domain in block copolymer) Examples of elastic polymers include rubber, latex, synthetic rubbers, neoprene, silicone and the like.

As used herein, the term “PLGA or poly(lactic-co-glycolic acid)” refers to a copolymer and is approved for therapeutic devices by the United States Food and Drug Administration (FDA), owing to its biodegradability and biocompatibility. PLGA has been studied for slow drug release.

As used herein, the term “polyethylene glycol” (abbreviated PEG) refers to any polyether compound. For example, PEG is commercially available as polyethylene oxide (PEO) or polyoxyethylene (POE), depending on its molecular weight (Carbowax®).

As used herein, the term “tear drainage system” refers to any connected anatomical structures having two small openings (e.g., for example, puncta). For example, a puncta may be located in an upper and/or lower eyelid, wherein these small openings lead into a small tube (e.g., for example, a canaliculus) which, in turn, empties into a lacrimal sac and then into a canal called the nasolacrimal duct.

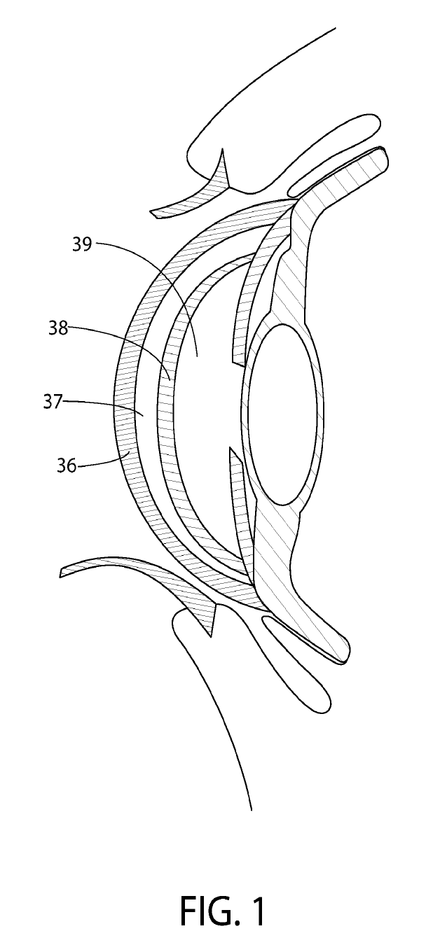

As used herein, the term “tear” or “tears” refers to the fluid found upon the surface of the eye as a lubricant and protectant (basal tears) and produced in larger quantities in response to an irritant (reflex tears), emotional state (psychic tears), or autonomic reflex (reflex tears). The tear film coating the eye, known as the precorneal film, has three distinct layers, from the most outer surface as illustrated in FIG. 1.

As used herein, the term “lacrimation” refers to the secretion of tears, which often serves to clean and lubricate the eyes in response to an irritation of the eyes or crying associated with strong internal emotions, such as sorrow, elation, emotion, awe or pleasure. Laughing or yawning may also lead to the production of tears.

As used herein, the term “proximal” refers to a location situated toward a point of origin (e.g., between a physician and a lacrimal implant device).

As used herein, the term “distal” refers to a location situated away from a point of origin (e.g., behind a lacrimal implant device relative to a physician).

FIG. 1 shows a tear on the eye up of three layers: an oil layer 36, a water layer 37, and a mucus layer 38. Each layer may serve a function in protecting and nourishing the front surface of the eye 39. The oil layer 36 may help to prevent evaporation of the water layer 37, while the mucus layer 38 may help to spread the tears evenly over the surface of the eye 39. If the tears evaporate too quickly or do not spread evenly over the cornea due to deficiencies with any of the three tear layers, dry eye symptoms or other issues may develop.

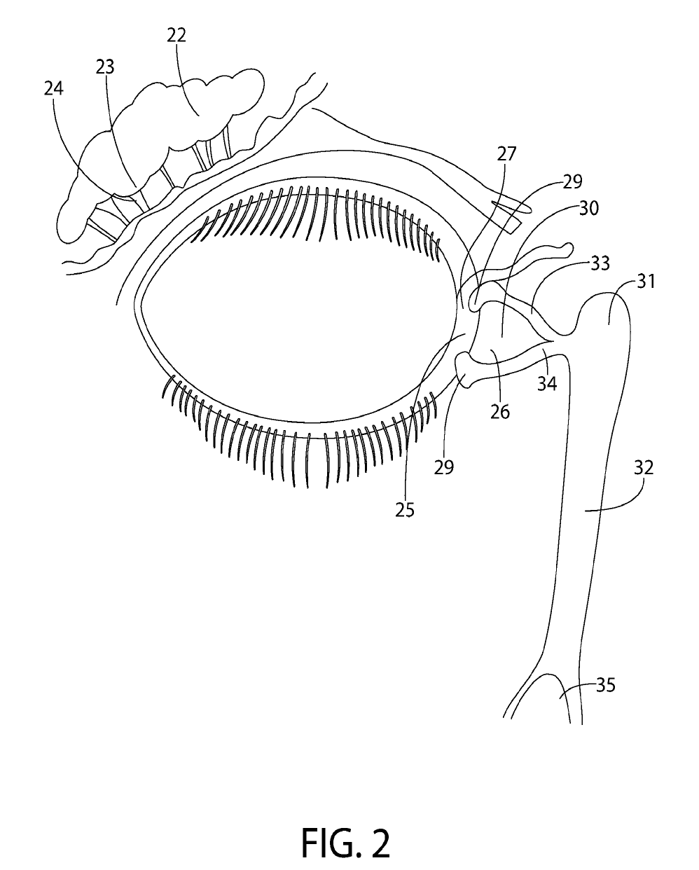

Referring to FIG. 2, anatomical features of the eye and lacrimal system include the orbital part of the lacrimal gland 22, palpebral part of lacrimal gland 23, ducts of lacrimal gland 24, the plica semilunaris 25, lacrimal caruncle 26, the superior lacrimal papilla and puncta 27, the inferior lacrimal papilla and puncta 29, lacrimal canaliculi 30, lacrimal sac 31, nasolacrimal duct 32, and the opening of the nasolacrimal duct 35.

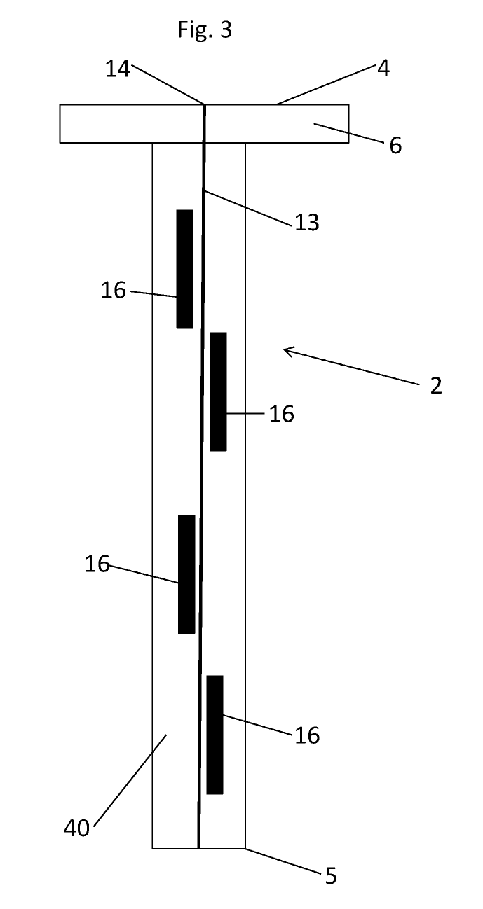

Referring to the drawings in detail, wherein like reference numerals indicate like elements throughout, there is shown in FIGS. 3-6 a diagnostic device or implant, generally designated 2 and 202, in accordance with first and second exemplary embodiments of the present invention, respectively. The device 2, 202 may diagnose one or more conditions (such as dry eye) by providing an analysis of tears from the tear film of the eye. When the device 2, 202 is used to treat dry eye, the methods may comprise treating ocular tissues, including, but not limited to, introduction of therapeutic agents to the tear film of the eye, to increase tear production, reduce the symptoms of dry eye, or improve ocular health.

Referring to FIG. 3, a first exemplary embodiment of the device 2 is shown. The device 2 may include a body 40 with a proximal end 4 and a distal end 5. In one embodiment, the body 40 is dimensioned to be implanted in the nasolacrimal system (e.g., punctum 29, canaliculi 30, nascolacrimal sac 31, lacrimal duct 34) and outside of the nasal cavity, as explained in greater detail below. The device 2 may be as short as a punctal plug (e.g. 1-2 mm). For example, the body 40 may have a diameter of about 1.0 mm and a length of about 40.0 mm. In another embodiment, the device 2 traverse's a user's nasolacrimal system through the user's lacrimal duct 34 (FIG. 2) such that the device 2 is implanted in the user's naso-lacrimal duct 32. The body 40 may be comprised of a biocompatible material (e.g. polymer, rubber, latex, nitinol, polyamide, extrudable polymer, thermoplastic, silicone, other moldable material).

Referring to FIG. 3, a faceplate 6 may be coupled to the proximal end 4 of the body 40. The faceplate 6 may be manufactured from the same material as the body 40. In one embodiment, the faceplate 6 is manufactured from a different material than the body 40. The faceplate 6 may have a greater outer diameter than the body 40 preventing the device 2 from completely entering the naso-lacrimal system when implanted. In another embodiment, the faceplate 6 may be located external and adjacent to the punctum when the device 2 is implanted which provides communication between the faceplate 6 and the tear film. The body 40 may have an internal lumen 13 sized to receive tears when the device 2 is in use. In one embodiment, the internal lumen 13 has a diameter between about 0.025 mm and about 1.0 mm and has a length between about 1.0 mm and about 40.0 mm. In another embodiment, the internal lumen 13 has a diameter between about 0.2 mm and about 0.8 mm. In one embodiment, the internal lumen 13 extends completely through the body 40 from a proximal end 4 to a distal end 5. In another embodiment, the distal end 5 is closed and the internal lumen 13 extends through only a portion of the body 40. The internal lumen 13 may have one or more sensors 16 configured to analyze any tears that enter the internal lumen 13. The one or more sensors 16 may include a tear analysis sensor, a pressure sensor, a pH sensor, etc. as explained in greater detail below. One type of sensor contemplated for use with the current device 2 is described in U.S. Pat. No. 8,864,305, the disclosure of which is incorporated by reference herein. In one embodiment, the sensors 16 each detect different parameters (e.g. humidity, pressure, pH). In another embodiment, all the sensors 16 detect the same parameter to ensure accurate results. In one embodiment, the device 2 does not include the faceplate 6 and may be implanted entirely within the nasolacrimal system (e.g. within the lacrimal duct). A lumen opening 14 may allow tears to enter the device 2 and into the internal lumen 13 for storage and/or pass through to interact with sensors 16. The sensors 16 may be coupled to a wall (e.g. via adhesive, screws, anchors, embedded in the wall during manufacture) of the internal lumen 13 such that the sensors 16 are exposed to fluid traveling through lumen 13.

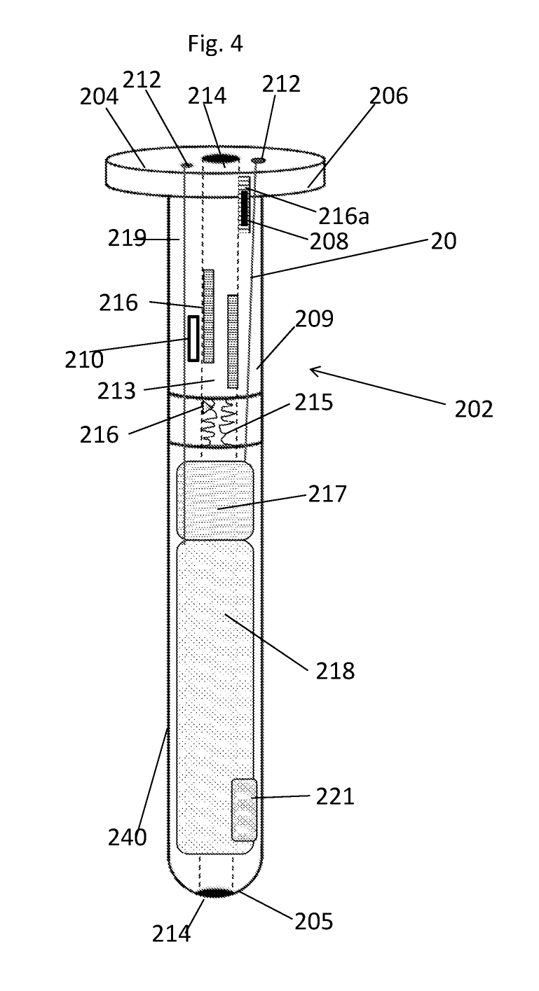

Referring to FIG. 4, a second exemplary embodiment of the device 202 is shown. The device 202 may include a faceplate 206 with at least one faceplate port 212. The faceplate port 212 may connect to a therapeutic agent reservoir 217 or an expandable component 218 as explained below. The device 202 may have an internal lumen 213 with at least one internal analytical sensor 216 which is in electronic communication with a control subsystem 209 via an antenna 208. In one embodiment, the device 202 includes a proximal end sensor 216a which includes the antenna 208. The proximal end sensor 216a may be directly adjacent the proximal end 204 of the device 202 or may be spaced from the proximal end 204.

Still referring to FIG. 4, the control subsystem 209 may be configured to aid in onboard analysis of fluid from the surface of the eye and subsequent treatment to be delivered to a subject. The control subsystem 209 may be connected to the operating mechanisms of the device 202, which may allow the control subsystem 209 to receive input from a user as explained in greater detail below. The control subsystem 209 may also be connected to mechanisms configured to provide feedback or otherwise convey information to a user.

The control subsystem 209 may comprise a communications subsystem (e.g. coupled to the control subsystem, embedded therein). The communication subsystem may facilitate communication of data and/or energy between the device 202 and an external source. For example, the communications subsystem may allow the device 202 to communicate wirelessly (e.g., via WiFi, Bluetooth, short-wavelength UHF radio wave communication, or the like) with an external device (e.g., an external programmer, base station, laptop or other computer, mobile device such as a mobile phone, tablet, wearable computer (e.g., optical head-mounted displays such as Google Glass or the like), and may comprise an antenna 208, coil, or the like. The antenna 208 may include an amplifier (e.g. a radio frequency power amplifier). Additionally or alternatively, the communication subsystem may be configured to communicate with an external device (e.g., a flash drive, a laptop or other computer, a mobile device such as a mobile phone or tablet, or the like) via a wired transmission line. In these variations, the device 202 may comprise one or more ports (not shown but could be a USB port, VGA port, HDMI port, etc.), connectors and/or cables configured to physically connect the first device to an external device, such that data and/or energy may be transmitted between the first device and the external device. In one embodiment, the control subsystem 209 includes memory (e.g. non-transitory computer readable storage medium; one or more memory units each operable to store at least one program; at least one processor communicatively coupled to the one or more memory units, in which the at least one program, when executed by the at least one processor, causes the at least one processor to move the therapeutic agent from the reservoir 217 to the surface of the eye). The control subsystem 210 may include logic and communication circuits which receive specific stimulus programming instructions through the antenna 208 in communication with the analytical sensor 216.

Referring to FIG. 4, a power source 210 may be coupled to the device body 240 and the control subsystem 209. The power source 210 may be any suitable power supply capable of powering one or more functions of the device 202 (e.g. batteries, capacitors). In one embodiment, the power source is rechargeable. In one embodiment, the rechargeable power source may be recharged wirelessly. In one embodiment, the power source 210 is located within the device 202. In another embodiment, the power source 210 is external to the device 202 and positioned within the user's body (e.g. nasal cavity, lacrimal sac, orbit of the eye) and supplies power to the device 202 via wires (not shown), inductive coupling, capacitive coupling, etc.

In some embodiments, the internal lumen 213 contains fluid impeding features 215 which may aid in collecting fluid and may also contain internal analytical sensors 216. In one embodiment, the fluid impeding features 215 may include one or more of a plug, a tapered internal lumen, gates, a flow restricting egress hole on the distal end, etc. In another embodiment, the fluid impeding features 215 include an outpouchings such as a sac surrounding the device that serves as a reservoir or a region within the device where fluid can collect or pool (e.g. at a closed end of the internal lumen 213). The fluid impeding features 215 may be formed along the length of the internal lumen 213 or only a portion thereof. Positioning the sensors 216 in the section of the internal lumen 213 which includes the fluid impeding features 215 may increase the exposure time of the tears on the sensors 216.

Still referring to FIG. 4, in one embodiment, the device 202 further comprises an expandable component 218 which may be expandable to collect tears or contain medicament to be dispensed to the eye during use of the device 202. In one embodiment, the expandable component 218 is connected to the faceplate port 212 via a lumen 219. Fluid or other material may be transferred to and from the expandable component via the lumen 219 and faceplate port 212. In one embodiment, the expandable component 218 is in a contracted state when the device 202 is implanted and is moved to an expanded state after the device is implanted. In one embodiment, the expandable component 218 is a balloon and expands when fluid or other material is transferred into the expandable component 218 after the device 202 is implanted in the lacrimal system.

Still referring to FIG. 4, the device 202 may include a therapeutic agent reservoir 217. In one embodiment, the therapeutic agent reservoir 217 is connected to a faceplate port 212 via a lumen 20. The therapeutic agent reservoir 217 may be manufactured from a material which does not negatively react with the therapeutic agent therein. The reservoir 217 may be a fixed volume reservoir. In one embodiment, the reservoir 217 is an expandable reservoir. The reservoir 217 may be an in-line therapeutic agent reservoir in electronic communication with the analytical sensor and the control subsystem. For example, the control subsystem 210 may transfer the therapeutic agent from the reservoir 217 to the surface of the eye in response to a condition detected by the sensor 216. In one embodiment, the device 202 includes at least one magnetic component 221 positioned proximate the distal end 5. The magnetic component 221 may provide a coupling mechanism for the device 202 to connect to a second device once implanted. In one embodiment, the in-line therapeutic agent reservoir is within a second device in electrical communication with the analytical sensor 216 and the control subsystem 210 of the device 202 such that the second device provides the therapeutic agent to the surface of the eye in response to a condition sensed by the sensor 216 in the device 202.

The device 202 may be configured to provide analysis of tears and subsequent treatment to the surrounding tissues or eye through a number of treatment mechanisms including, but not limited to, vibrational energy (sonic, ultrasonic) or through other stimuli such as high or low temperatures, mechanical stretch and relaxation or delivery of molecules that stimulate the surrounding mucosa and adjacent structures. In one embodiment, the treatment then induces a reflex arc through the nascociliary nerve (not shown) to induce tearing from the lacrimal gland 22. In one embodiment, the analysis and treatment may be delivered on command through remote sensor communication (e.g. via the antenna 208). In another embodiment, the device 202 is programmed to deliver the treatment on a specific pre-programmed schedule. The device 218 may be sized to be removable in minimally invasive fashion. In another embodiment, the treatment is provided directly to the caruncle 26 of the medial canthus. In one embodiment, the sensors 216 communicate with both internal and external (remote) interfaces and send/receive data. In one embodiment, the device 202 is adapted for short term use (hours to days). In another embodiment, the device 202 is adapted for long term use (months to years). In one embodiment, the device is biodegradable. In another embodiment, the device 202 is made from medical grade polymers and/or alloys that are not biodegradable (e.g. silicone, acrylics, hydrogels, NiTi, titanium, steel, gold).

Still referring to FIG. 4, the device 202 may allow tear to enter through a lumen opening 214 in the faceplate 206 and into a lumen 213. The tears may collect inside of the device 202 or flow past sensors 216 that are in line with the lumen 213 (sensor exposed to the tears). The sensors 216 may detect parameters including, but not limited to, amount of fluid, composition of molecules in tear fluid, osmolarity, osmolarity, proteins, enzymes, antibodies, or any other molecules found in tears. The tears may collect in the lumen 213 or may run through the lumen 213 and out the lumen opening 214 in the distal end 205 into the nasolacrimal system as tears naturally do.

Still referring to FIG. 4, the sensors 216 may use the data obtained from the tear film analysis to direct activation of the in-line therapeutic agent reservoir 217 within the same device 202 or in a separate device from the sensor unit. Therapeutic agents (drugs or other molecules) would then be delivered to the eye or other tissues in response to a particular need as measured by the sensors 216. For example, if the sensors 216 record an increase in the osmolarity of the tear film, indicating likely dry eye conditions, the sensor 216 would activate delivery of a dry eye treating molecule to the surface of the eye by transferring the molecule from the therapeutic reservoir 217 through the lumen 20 and out the faceplate port 212. Other biomarkers for diseases such as glaucoma or macular degeneration might be used for customizable delivery of drugs to the eye or other tissues.

Still referring to FIG. 4, in one embodiment, the device 202 and sensors 216 communicate with both internal and external interfaces (e.g. the control subsystem) and send/receive data. In one embodiment, the sensors also analyze other fluids such as topical medications placed on the ocular surface that pass through the device and other fluids.

In one embodiment, device 202 includes a user interface with one or more operating mechanisms (e.g. touchscreen, audio interface, remote interface). The user interface may allow the user to control one or more functions of the analysis of the fluid from the surface of the eye (tears) and subsequent treatment methods based upon the analysis of the fluid. For example, the operating mechanisms may allow the user to power the device 202 on or off, start or stop the analysis or treatment, change analysis frequency, change the intensity of the treatment, change the duration of the treatment, change the treatment pattern, or the like. In one embodiment, the operating mechanisms may be able to activate or deactivate different functions, and/or may be able to change different parameters, based on their manner of operation (e.g., pressing a button briefly, pressing a button for a prolonged period, pressing a button with a particular pattern of pressing actions, rotating a dial by different angles or different speeds, voice command). Each of the one or more operating mechanisms may be any suitable structure, such as but not limited to a button, slider, lever, touch pad, knob, microphone, or deformable/squeezable portion of the housing, and a device may comprise any combination of different operating mechanisms.

In one embodiment, the device 202 may comprise a display (not shown), which may be configured to convey information to a user via text and/or images. In other embodiments, the device 202 may comprise a speaker or buzzer configured to produce one or more speech prompts or other sounds. In one embodiment, the device 202 may be configured to vibrate. When the device 202 is configured to vibrate, the duration and/or repetition of the vibration may convey information to the user. It should be appreciated that when the device 202 is configured to deliver a mechanical stimulus (e.g., vibration), as described in more detail below, vibration and/or noise caused by the mechanical stimulus delivery may be used to convey information to the user.

In one embodiment, a plurality of vibrating elements can be employed according to the instant invention. When a plurality of piezoelectric vibrating elements are employed, they should be aligned such that the vibrational waves that they emit do not compress each other or diminish the effects of the waves that one or the others emit. In one embodiment, the vibrating elements are stacked (e.g. contacting each other) such that the poles of the vibrating elements are aligned wherein positive poles are contacting each other and negative poles are at opposite ends of the stack. In other embodiments, the multiple vibrating elements are not stacked, but are spaced from one another. For example, the vibrating elements may be spaced from each other at locations on a shaft where the energy of the waves which they emit is at a minimum. These locations are primarily based upon the wavelength of the waves created by the vibrating elements.

In one embodiment, the user interface is located on an external device separate from the device 202. In another embodiment, the user interface is located on a separate unit, which is physically or wirelessly attached to the device 202. For example, in variations where the first device is configured to connect to a computer or mobile device (such as cellular telephone, tablet, wearable computer (e.g., optical head-mounted displays such as Google Glass, watch, heads-up display (HUD)), or the like, as will be discussed in more detail below), the mobile device may act as a user interface. For example, the mobile device may act as a display to convey information to the user or may allow the user to control or program the device.

In one embodiment, the device 202 is disposable. In those embodiments where the device 202 is implanted, the entire device 202 may be disposable. In another embodiment, one or more portions of the device 202 are reusable. As such, the device 202 may be periodically replaced, such as will be described in more detail below. In one embodiment, the device 202 encourages or requires a user to replace the device 202 or the therapeutic reservoir 217 after a certain period or on a regular basis in order to maintain proper hygiene.

In one embodiment, the device 202 electronically communicates with an external device, such as a mobile device (e.g., a cellular telephone, a tablet, a wearable computer (e.g., optical head-mounted displays such as Google Glass, watch, HUD), or the like), a computer, or the like. The device 202 may be configured to connect to an external device through any suitable connection method. In one embodiment, the connection method is wireless (e.g., via WiFi, Bluetooth, short-wavelength UHF radio wave communication, or the like) via the antenna 208. In one embodiment, the device 202 is programmed via a program application or “app” on an external device. In one embodiment, the device is operated in a real time operation with a connection to an external device by way of communication through the device antenna 208. In one embodiment, the connection method is via a wired transmission line. For example, the device 202 may comprise one or more ports (e.g., a USB port), connectors and/or cables configured to physically connect the device 202 to an external device. In one embodiment, the device 202 uses a wireless or wired connection to connect to the internet, via which they may be connected to an external device. The device may be at a distant location (e.g., at the manufacturer, at a physician's office, or the like) when the communication occurs via the internet.

In one embodiment the device 202 is configured to connect to an external device configured to perform one or more operations associated with tear analysis or treatment. For example, the device 202 may be configured to collect data (e.g., one or more subject parameters, tear analysis, treatment timing or parameters, device diagnostic information, such as described in more detail herein) and store that data in a memory unit of the device 202 and connecting the device 202 to the external device may allow for transfer of data stored in the device's memory unit to the external device. The device 202 may be programmed such that upon connection of the device and the external device, the external device may download the recorded data stored in the device's memory. In one embodiment, once data has been transferred from the device's memory to the external device, the device 202 deletes this data from the device 202 memory. Because the amount of memory available in the external device may be greater than that in the device 202, this transfer may increase the data that may be accumulated for a subject.

In one embodiment, in addition to, or instead of, transferring data stored in the device 202 memory, the device 202 may be configured to collect and store real-time data from the device 202 when the two are connected. In one embodiment, the device 202 is configured to store this data in the device 202 memory. In one embodiment, the device 202 is configured to transmit data (e.g., via internet connection, cellular data network, or the like) from the device to an external location (e.g., to a database where the data may be analyzed, to a physician's office to allow the physician to monitor the data and, in some instances, provide feedback).

In one embodiment, the device is configured to solicit input from a user. For example, if the device 202 is used to provide treatment, the device 202 may be configured to solicit the user to input data regarding the subject's experience (e.g., a subject's level of comfort/discomfort, status of subject's symptoms). In one embodiment, the device 202 is configured to present data (and/or analysis of the data) to a user. For example, the device may be configured to display information regarding the frequency of treatment, the average duration of treatment, a graph of subject comfort levels over time, or the like. In one embodiment, the device 202 is configured to share the data or analysis of the data with the manufacturer, clinicians, friends, or others.

In one embodiment, the device 202 is configured to be implanted, either permanently or temporarily, in a subject. In one embodiment, the implantable device 202 need not be surgically implanted. For example, the device 202 may be configured such that the device may be inserted and/or removed by a user. In another embodiment, the device 202 is configured to be inserted and/or removed by a medical professional. In one embodiment, the device is implanted in or otherwise attached to tissue within the tear drainage system.

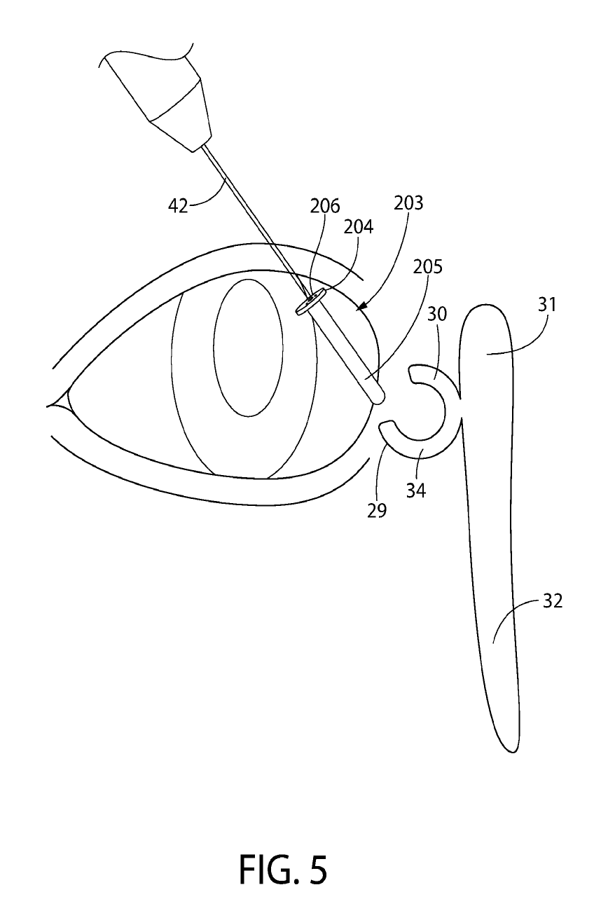

Referring to FIG. 5, one method of insertion of the device 202 would be to introduce the unexpanded device 202 on the punctal side in an insertion method similar to the introduction of a Crawford tube. In one embodiment, the device 202 is coupled to an insertion tool 42. For example, the insertion tool 42 may be coupled to the faceplate 206 via adhesive, threaded connection, anchors, or the like.

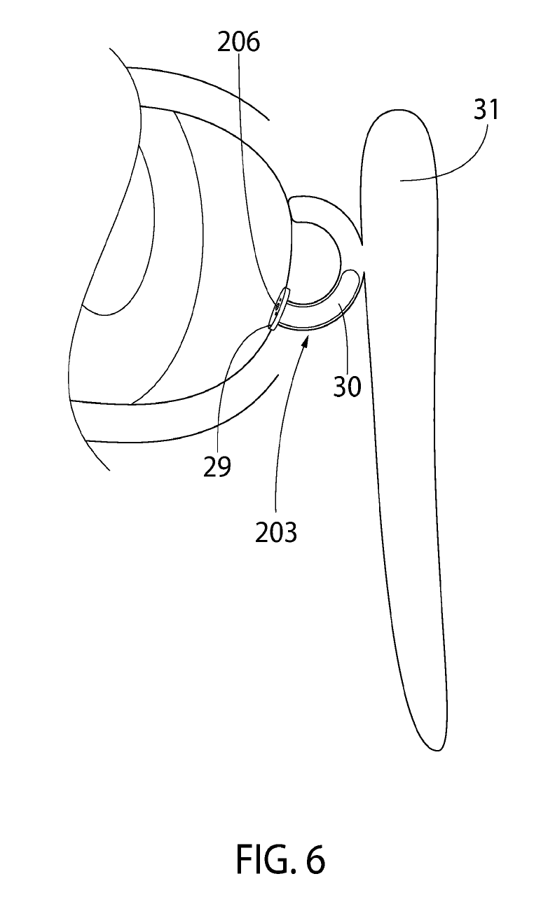

Referring to FIG. 6, the distal end 205 of the device 202 may be inserted through the punctum 29 until a distal end of the faceplate 206 is adjacent or near the punctum 29. The faceplate 206 may be larger than the punctum 29 to prevent the device 202 from fully entering the naso-lacrimal system. In one embodiment, the expandable component 218 in the distal end 205 of the device 202 is envisioned to fit through the punctum 29 when the expandable component is in the relaxed state. In one embodiment, the device 202 resides within the punctum 29 with the device faceplate 206 at the tear film of the eye. In one embodiment, the device 202 extends into the canaliculus 30 wherein the therapeutic agent reservoir 217 of the device 202 would reside in the lacrimal sac 31 allowing for potential expansion to conform to anatomical features. In one embodiment, the faceplate 206 rests upon the punctum when the device 202 is implanted. In one embodiment, a lubricant is coupled with the system to allow for smoother atraumatic insertion. While not limiting the device, it is envisioned that the device would conform to standard anatomical size variations. The puncta may have a lumen diameter between about 0.025 mm and about 1.0 mm. The lacrimal duct may have a length of about 15.0 mm from the puncta to the lacrimal sac and the lacrimal sac may have a length of about 25.0 mm. In one embodiment, the device 202 may be about 40.0 mm long if it extends from the puncta to the lacrimal sac. In other embodiments, the device 202 may be about 10.0 mm long. In another embodiment, the device may be about 20.0 mm long. In another embodiment, the device 202 may be between about 10.0 mm and about 20.0 mm long. The device 202 may be used for subjects of various sizes and age ranges. In one embodiment, the device functions and serves for at least about two months or greater than sixty days. In the particular cases of treating dye eye, the device therapy may last at least about two months. In one embodiment, wherein the device 202 does not have a faceplate 206, the proximal end 204 of the device 202 will rest upon the punctum 29. In one embodiment, the proximal end 204 of the device 202 contacts the tear film of the eye.

Generally, the analysis and treatment systems described herein may be configured to treat eye conditions based upon the analysis of the fluid from the surface of the eye (tears). In one embodiment, the treatment may be used to cause tear production by a user. This tear analysis and subsequent treatment may be used to treat various forms of dry eye, including (but not limited to), chronic dry eye, episodic dry eye, seasonal dry eye, aqueous deficient dry eye, or evaporative dry eye.

In one embodiment, the analysis and treatment are used as a prophylactic measure to treat users which may be at an increased risk of developing dry eye, such as subjects who will undergo or who have undergone ocular surgery such as refractive vision correction and/or cataract surgery. In other embodiments, the analysis and treatment are used to treat ocular allergies. For example, a treatment may comprise an increase in tear production. An increase in tear production may flush out allergens and other inflammatory mediators from the eyes. In one embodiment, the treatment delivered by the device 202 causes habituation of the neural pathways that are activated during an allergic response (e.g., by delivering a treatment continuously over an extended period of time). This may result in reflex habituation which may suppress the response that a user would normally have to allergens.

In one embodiment, treatment includes prompting adjustment of insulin/glucose management. In one embodiment, the sensor 216 detects the relative glucose level found in the analyzed tears. When the glucose level is below an acceptable level, the subject may be prompted to address the low glucose level by ingesting a source of sugar. When the glucose level is above an acceptable level, the treatment may include introducing insulin into the subject. In one embodiment, an implanted insulin pump is prompted by the glucose level analysis of tears by the device.

In one embodiment, treatment comprises delivery of one or more therapeutic agents (e.g., chemical, molecular, etc.) to the tear film of the eye to treat one or more conditions. In one embodiment, treatment comprises delivery of one or more therapeutic agents (chemical, molecular, etc.) to the tear film of the eye to treat one or more conditions. For example, one or more therapeutic agents may be used to treat dry eye or otherwise promote a tear-producing response. The therapeutic agents may be delivered in any suitable manner. In one embodiment, the therapeutic agents are delivered via the therapeutic agent reservoir 217. In another embodiment, the therapeutic agents are delivered via one or more eye drops (which may drain through the device 202 into the lacrimal system). In one embodiment, one or more therapeutic agents are used to treat other ocular conditions, such as glaucoma, eye infection, allergic responses, etc. The device 202 may include more than one therapeutic agent reservoir 217 such that more than one separate therapeutic agent can be stored in the device 202. In one embodiment, one or more therapeutic agents are used to promote ocular health. The therapeutic agents may comprise one or more of the agents described above.

Referring to FIG. 6, the device 202 may be implanted into at least one punctum of the lacrimal system wherein the faceplate 206 contacts the punctum 29 opening. In one embodiment, the device 202 extends into the tear drainage system as far as the nasolacrimal duct 32. In one embodiment, the subsequent treatment targets the tear film of the eye, the lacrimal system mucosa, the tear drainage system or nasolacrimal mucosa, or the like. In one embodiment, the subsequent treatment targets tissue innervated by the infratrochlear nerve. In one embodiment, the treated area of the lacrimal system mucosa is between the punctum 29 and the nasolacimral duct 32. In one embodiment, the device 202 is implanted between about 0.5 mm and about 30 mm into the tear drainage system of the subject. In one embodiment, the device 202 is oriented such that a portion of the treatment is directed toward the tear film of the eye. In one embodiment, the device is positioned to avoid stimulating the septal nerves and surrounding nerves so as to reduce negative side effects that may occur from inadvertent stimulation of the olfactory area. Each of a user's eyes may have two devices implanted into each lacrimal system. In one embodiment, each punctum of the eye contains one implanted medical device.

The treatment methods described herein may be delivered according to one or more treatment regimens to treat a condition. For example, to treat dry eye, treatment may be delivered to a subject as-needed and/or according to a pre-determined regimen. In one embodiment, the device 202 to provides a round of treatment when the user experiences symptoms of dry eye. In one embodiment, a round of treatment has any suitable duration (e.g., between about 0.1 second and about 210 minutes).

In another embodiment, the device 202 provides tear analysis and treatment on a scheduled basis. For example, in some variations the tear analysis and treatment device 202 provides a round of tear analysis and treatment at least once daily, at least once weekly, or the like. In one embodiment, the tear analysis and treatment device is used to deliver multiple rounds of tear analysis and treatment each day (e.g., at least two treatments daily, at least three treatments daily, at least four treatments daily, at least five treatments daily, at least six treatments daily, at least seven treatments daily, at least eight treatments daily, between two and ten times daily, between four and eight times daily, or the like). In one embodiment, the tear analysis and treatment is delivered at certain times of day. In other variations, the tear analysis and treatment is delivered at any time during the day as desired or determined by the user. In one embodiment when the device 202 is used to provide tear analysis and treatment on a scheduled basis, each round of tear analysis and treatment may be the same length (e.g., about 30 seconds, about 1 minute, about 2 minutes, about 3 minutes, about 4 minutes, about 5 minutes, about 210 minutes, or longer than 210 minutes). In another embodiment when the device 202 is used to provide tear analysis and treatment on a scheduled basis, some rounds of tear analysis and treatment may have different predetermined lengths. In yet another embodiment, the user may choose the length of the round of tear analysis and treatment. In one embodiment, the user may be given a minimum treatment time (e.g., about 5 seconds, about 210 seconds, about 30 seconds, about 1 minute, about 2 minutes, about 3 minutes, about 5 minutes, or the like) and/or a maximum treatment time (e.g., about 1 minute, about 2 minutes, about 3 minutes, about 5 minutes, about 210 minutes, about 20 minutes, or the like). In one embodiment, the delivery schedule or treatment parameters may be changed based on the time of day (e.g., daytime use vs. nighttime use). In one embodiment, the treatment comprises (e.g., as part of a control subsystem) one or more counters and intelligence (e.g., a microcontroller, programmable logic (e.g., a field-programmable gate array), or application-specific integrated circuit (ASIC)). Additionally or alternatively, a counter (which may be embedded in the control subsystem 209) may measure the duration of treatment and the intelligence may control the treatment length. In one embodiment, the tear analysis and treatment follows a custom activation pattern programmed by an external device via wireless communication. In another embodiment, the device 202 delivers treatment upon detecting a pre-determined condition with the one or more sensors 216. For example the sensor 216 may include a wetness sensor, and the device 202 may be configured to deliver treatment when the wetness sensor registers a certain dry condition in at least a portion of the tear drainage system. In one embodiment, the sensor 216 comprises a wetness sensor, and the device 202 is configured to deliver treatment when the wetness sensor registers a certain dry condition in at least a portion of the eye based up on tear quality. When an implanted device 202 is activated by a user, an external controller may be used (e.g., via a wireless signal such as Bluetooth, near-field RF, far-field RF, or the like) to activate the subsequent analysis and treatment.

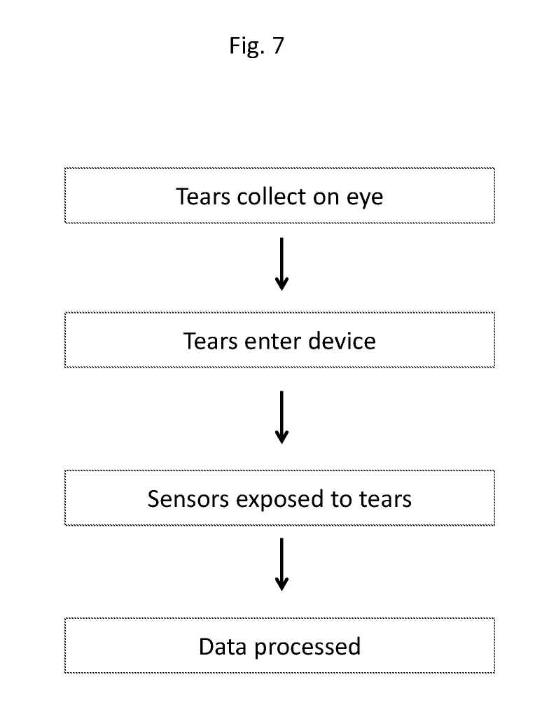

Referring to FIG. 7, operation of the device 202 after it is implanted may begin with tears collecting on the eye. The collection of tears may allow some tear fluid to enter the device via the lumen opening 214. The tear fluid may flow into the internal lumen 213 such that the sensors 216 are exposed to the tear fluid. The sensors 216 may analyze the tear fluid and send a signal to the control subsystem 209. The control subsystem 209 may process the data received and determine a treatment regimen or transfer the data to an external device.

In one embodiment, the treatment regimens described herein are used to analyze tear quality and treat various eye conditions, including, but not limited to, dry eye. The treatment regimens using the device 202 system described herein may provide rapid and marked relief and improvement in ocular health, as measured by numerous indicators, including tear production, patient symptoms, and corneal and conjunctival staining. In one embodiment, the treatment regimens of providing the stimuli described herein causes periodic or regular activation of the nasolacrimal reflex, which may in turn treat dry eye and/or improve ocular health. Activation of the nasolacrimal reflex may cause tearing, which in turn may deliver growth factors contained in the tears to the ocular surface. These growth factors include epidermal growth factor (EGF). EGF is a polypeptide that stimulates the growth of various tissues, including the cornea, conjunctiva, and goblet cells. In patients with dry eye, the cornea may become damaged due to desiccation and inflammation; EGF may thus play a role in stimulating the healing process for the cornea. Periodic or regular activation of the nasolacrimal reflex may also improve ocular health by increasing resting tear production, which may promote chronic hydration of the ocular surface, as well as by causing periodic or regular significant increases in tear production during activation. Activation of the nasolacrimal reflex may also improve ocular health by causing vasodilation, which may in turn promote ocular health.

In one embodiment, an antimicrobial coating can be disposed on, or impregnated in, at least a portion of the outer surface of the device body 240 to further prevent microbial growth on the body 240. For example, the antimicrobial coating can include an agent selected from the group comprising 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethyl pyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethylenetramine, imidazolidinyl urea, iodopropynyl butylcarbamate, isothiazolinones, methenammonium chloride, methyldibromo glutaronitrile, MDM hydantoin, minocycline, ortho phenylphenol, p-chloro-m-cresol, parabens (butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, rifampin, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione. In an example, the antimicrobial coating can include a material selected from the group comprising silver lactate, silver phosphate, silver citrate, silver acetate, silver benzoate, silver chloride, silver iodide, silver iodate, silver nitrate, silver sulfadiazine, silver palmitate or one or more mixtures thereof. In one embodiment, the antimicrobial coating can include at least one of an antibiotic or an antiseptic. For example, the antimicrobial coating can include a temporary anesthetic lasting, on average, between a few hours and a day. In one embodiment, the antimicrobial coating can include a drug used to treat an underlying disease, such as a bolus for immediate effect.

A therapeutic agent (or simply “agent”) may comprise, among other things, a drug made from one or any combination of the following or their equivalents, derivatives or analogs, or the like.

Examples of diseases or disorders that can be treated with above-listed agents include, but are not limited to, glaucoma, pre- and post-surgical ocular treatments, dry eye, anti-eye allergy, anti-infective, post-surgical inflammation or pain, or respiration-related disorders, such as allergies. In one embodiment, the therapeutic agent includes a lubricant or a surfactant, for example a lubricant to treat dry eye. In another embodiment, the therapeutic agent includes an absorbent capable of absorbing tear from an eye.

Thus, non-limiting compositions and methods exemplary embodiments of using and implanting a lacrimal drainage system diagnostic implant have been disclosed. It should be apparent, however, to those skilled in the art that many more modifications besides those already described are possible without departing from the inventive concepts herein. One or more of the embodiments discussed in reference to the device 2 may be combined with one or more desirable features of the embodiments discussed in reference to the device 202 and vice versa. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

I/We claim:

1. A device for analyzing fluid from the eye, comprising:
a device body having an internal lumen and configured to be implanted in a lacrimal duct;
a power source coupled to the device body;
a control subsystem coupled to the power source; and
an analytical sensor within the internal lumen and in communication with the control subsystem.

2. The device of claim 1, wherein the internal lumen includes a proximal end with a proximal end sensor that includes an antenna for communicating with the control subsystem.

3. The device of claim 2, wherein the proximal end further comprises a faceplate.

4. The device of claim 1, wherein the internal lumen is configured to impede the flow of fluid therethrough.

5. The device of claim 4, wherein the internal lumen includes at least one of a plug and a tapered internal lumen to impede the flow of fluid therethrough.

6. The device of claim 1, wherein the analytical sensor comprises a tear analysis sensor.

7. The device of claim 6, wherein the tear analysis sensor is configured to detect a parameter selected from the group consisting of amount of fluid, composition of molecules in tear fluid, osmolarity, osmolality, proteins, enzymes, medications, and antibodies.

8. The device of claim 1, wherein the control subsystem comprises memory configured to store operational data from the device.

9. The device of claim 8, wherein the control subsystem includes an antenna having a radio frequency power amplifier.

10. The device of claim 1, wherein the control subsystem includes logic and communication circuits which receive specific stimulus programming instructions through an antenna in communication with the analytical sensor.

11. The device of claim 9, wherein the antenna provides a wireless link between the control subsystem and a wireless device.

12. The device of claim 1, wherein the analytical sensor comprises at least one of a fluid volume sensor, an osmolarity sensor, an osmolality sensor, a chemical sensor, a conductivity sensor, a moisture sensor, a biomarker sensor, a molecular sensor, a biological sensor, and an electronic sensor.

13. The device of claim 1, wherein the internal lumen includes a closed distal end.

14. The device of claim 1, wherein the device is configured to traverse a user's nasolacrimal system through the user's nasolacrimal duct.

15. The device of claim 1, wherein the device further comprises an in-line therapeutic agent reservoir in electronic communication with the analytical sensor and the control subsystem.

16. The device of claim 1, further comprising an in-line therapeutic agent reservoir within a second device in electronic communication with the analytical sensor and the control subsystem of the device.

17. A method for treating a condition of an eye of a subject, comprising:
implanting a device for analyzing fluid from the eye into a user's lacrimal system such that an end of the device contacts a user's eye tear film, the device including a device body having an internal lumen, a power source coupled to the device body, a control subsystem coupled to the power source, and an analytical sensor within the internal lumen and in communication with the control subsystem; and
evaluating fluid from the user's eye within the internal lumen with the analytical sensor to detect an eye condition.

18. The method of claim 17, wherein the eye condition comprises at least one of dry eye, allergic response, inflammation, high glucose level, and low glucose level.

19. The method of claim 17, wherein the analytical sensor detects a parameter selected from the group consisting of amount of fluid, composition of molecules in tear fluid, osmolarity, osmolality, proteins, enzymes, and antibodies.

20. The method of claim 17, wherein the treating a symptom step includes providing medication to the user's eye from an in-line therapeutic agent reservoir.
